(19) United States
(12) Patent Application Publication
(10) Pub. No.: US 2017/0105499 A1
(43) Pub. Date: Apr. 20, 2017

(54) DECORATIVE ACCESSORY FOR A MOBILE DEVICE

(71) Applicant: Anita Imbesi, Wellington, FL (US)

(72) Inventor: Anita Imbesi, Wellington, FL (US)

(21) Appl. No.: 14/942,365

(22) Filed: Nov. 16, 2015

Related U.S. Application Data
(63) Continuation-in-part of application No. 14/882,713, filed on Oct. 14, 2015.

(51) Int. Cl. A45C 13/00; A41G 1/00; A61L 9/12; F16M 11/38

(52) U.S. Cl. CPC A45C 13/001; F16M 11/38; A41G 1/00; A61L 9/12

(57) ABSTRACT

A decorative accessory, having a substantially tubular-shaped portion, a decorative element, and at least one cosmetic item. The tubular-shaped portion has an outer surface, an inner surface, and a cavity bounded by the inner surface. The decorative element has an extension member extending downwardly therefrom, where the extension member is secured to the tubular-shaped portion. The at least one cosmetic item is disposed within the cavity. The at least one cosmetic item includes a container, a cap, and a fluid medium disposed within the container.

### CROSS-REFERENCE TO RELATED APPLICATIONS

This application is a Continuation-in-Part patent application under 35 U.S.C. §120 of U.S. patent application Ser. No. 14/882,713 filed Oct. 14, 2015, which application is incorporated herein by reference.

### FIELD OF THE INVENTION

The invention broadly relates to a decorative accessory for a mobile device, and in particular, to a decorative accessory removably secured to a case for a mobile device, such as a cellular phone, tablet, etc.

### BACKGROUND OF THE INVENTION

Mobile devices are well known in the art and have been available since the mid-1980's when the first cellular phone arrived on the market. Thus a need for cases, which are designed to attach to, support or otherwise hold a cellular phone in order to protect it from damage, also evolved. Presently, there are a wide variety of types of mobile device cases available, such as pouches and sleeves, holsters, shells, skins, bumpers, and flip cases and wallets, etc. One problem with prior mobile device cases is that while many of these cases are customizable, to an extent, they lack a removable decorative accessory that has a multifaceted use as well.

For example, Lin discloses in U.S. Patent Application Publication No. 2014/0357330, a case for an electronic device having an interchangeable decorative element. Lin teaches the use of a cellular phone case having a decorative element on the back surface. Similar to Lin, Wang et al. disclose a case for a mobile phone having a floral surface pattern. Wang et al. teach the use of a cellular phone case having a decorative pattern on the back surface.

In U.S. Patent Application Publication No. 2014/0162735, Oh et al. disclose a case for a mobile device having apertures for both a touch pen and writing pen disposed therein. Oh et al. teach the use of a cellular phone case having an aperture with an element, in this case, a pen, removably disposed therein. Similar to Oh et al., Chang discloses a case for a mobile device having an aperture for a writing instrument disposed therein. Chang teaches the use of a cellular phone case having an aperture with an element, in this case, a writing instrument, removably disposed therein.

As mobile devices have evolved over time and become commonplace in everyday activities, customization has become increasingly important. Out of a need for personalization, cases having decorative accessories have evolved. Currently, there are vast assortment of cases available. One problem with present cases is that the decorative element is usually incorporated within or embedded within the case itself. Another problem with present cases is that the case and/or decorative element is integral with or fixedly secured to the case and is not removable. Yet another problem with present cases is that the decorative element has limited functionality beyond merely being a decorative object.

Thus, there is a long-felt need for a decorative accessory removably secured to a mobile device case. Additionally, there is another long-felt need for a decorative accessory having a cosmetic item, such as lip-gloss, mascara, etc. disposed within the accessory.

Moreover, another problem with present mobile device cases are that most are limited to solely protecting the phone without other functions. Thus, there is a long felt need for a decorative accessory secured to a mobile device case that not only provides a decorative element but that also appeals to other senses, such as olfactory senses as well. Similarly, there is another long felt need for a decorative accessory for a mobile device case that can also hold a fragrance, such as a perfume or essential oil, as essential oils are thought to provide a variety of health benefits. Furthermore, there is also a long felt need for a decorative accessory having fragrances that can be removed and replaced as the fragrance is used.

### BRIEF SUMMARY OF THE INVENTION

The present invention broadly includes a decorative accessory having a substantially tubular-shaped portion, a decorative element, and at least one cosmetic item. The tubular-shaped portion has an outer surface, an inner surface, and a cavity bounded by the inner surface. The decorative element has an extension member extending downwardly therefrom, where the extension member is secured to the tubular-shaped portion. The at least one cosmetic item is disposed within the cavity.

In one embodiment, the decorative accessory further includes a stand. The stand has a mount, a flange, and at least one hinge, where the stand extends laterally from the tubular-shaped portion. The mount is secured to a mobile device or mobile device case and the flange is secured to and pivotable outwardly about the at least one hinge. The decorative accessory further includes an attachment means to secure the extension member of the decorative element to the tubular-shaped portion. Additionally, the decorative element is substantially flower-shaped.

The at least one cosmetic item includes a container, a cap, and a fluid medium disposed within the container. In particular, the decorative accessory includes two cosmetic items disposed opposite one another within the cavity. Preferably, the fluid medium is lip-gloss, lipstick, concealer, mascara, etc. However, it should be appreciated that the fluid medium may be any suitable medium known in art.

In another embodiment, the present invention broadly includes a decorative accessory having a substantially tubular-shaped portion, a decorative element, at least one cosmetic item, and a clip. The tubular-shaped portion has an outer surface, an inner surface, and a cavity bounded by the inner surface. The decorative element has an extension member extending downwardly therefrom, where the extension member is secured to the tubular-shaped portion. The at least one cosmetic item is disposed within the cavity. The clip have a first extension, a second extension, a first handle, a second handle, and a hinge, where when the second handle is depressed, the second extension pivots outwardly into an open position. The clip is securable about a device when the second extension is released and pivots inwardly into a closed position.

Similar to the first embodiment, the decorative accessory further includes an attachment means to secure the extension member of the decorative element to the tubular-shaped portion. Additionally, the decorative element is substantially flower-shaped. The at least one cosmetic item includes a container, a cap, and a fluid medium disposed within the container. In particular, the decorative accessory includes two cosmetic items disposed opposite one another within the cavity. Preferably, the fluid medium is lip-gloss, lipstick, concealer, mascara, etc. However, it should be appreciated that the fluid medium may be any suitable medium known in art.

Preferably, the decorative accessory is made of plastic and substantially tubular-shaped. However, it should be appreciated that any of the elements can be made of any suitable material known in the art and can be of any suitable shape known in the art.

In yet another embodiment, the decorative element broadly includes a housing, a fragrance disc, and a means for securing the decorative element to a mobile device or a case for a mobile device. The housing has a top, a base, and an attachment means. The attachment means secures the top to the base and the top is pivotable about the attachment means. The base has an outer surface, an inner surface, a side and a cavity bounded by the side and the top includes a rim and at least one aperture bounded by the rim. The fragrance disc has an outer surface and an inner surface and is disposed within the cavity bounded by the side. The fragrance disc rests upon the inner surface of the base and the fragrance disc is filled with a scented medium. Preferably, the attachment means that secures the top to the base is a hinge. Preferably, the means for securing the decorative element to a mobile device is an extension member extending downwardly therefrom.

In another embodiment, the housing is substantially circular in shape and the top is convex. Preferably, the decorative element is substantially flower-shaped. However, it should be appreciated that any of the elements can be made of any suitable material known in the art and can be of any suitable shape known in the art.

In yet another embodiment, the top further includes at least one barb extending upwardly therefrom, and the decorative element further includes a flower ovary and a plurality of petals. The flower ovary is secured to the at least one barb and the plurality of petals are disposed circumferentially about the base. Preferably, the scented medium is a perfume or an essential oil. However, is should be appreciated that the scented medium can be made of any suitable medium known in the art. Essential oils, such as lavender, have been thought to promote health benefits, such as soothing nervous tension, headaches, anxiety and depression, inducing sleep, relieving pain from soreness in the joints, disinfecting the scalp and skin, enhancing blood circulation, treating respiratory problems, and increasing the mobility of the intestine, as well as a variety of other health benefits.

These and other objects and advantages of the present invention will be readily appreciable from the following description of preferred embodiments of the invention and from the accompanying drawings and claims.

### BRIEF DESCRIPTION OF THE DRAWINGS

The nature and mode of operation of the present invention will now be more fully described in the following detailed description of the invention taken with the accompanying drawing figures, in which:

FIG. 1 is a front perspective view of a first embodiment of a decorative accessory for a mobile device case;

FIG. 2 is a front perspective view of the accessory of FIG. 1, shown with the case removed;

FIG. 3 is a front elevational view of the accessory of FIG. 2;

FIG. 4 is a rear elevational view of the accessory of FIG. 2;

FIG. 5 is a cross-sectional view of the accessory, taken generally along line 5-5 of FIG. 4;

FIG. 6 is a detailed view of a first container of FIG. 5;

FIG. 7 is a detailed view of a second container of FIG. 5;

FIG. 8 is a second embodiment of the decorative accessory, shown having a clip attached and with the mobile device case removed;

FIG. 9 is an exploded view of an embodiment of a decorative element, shown having a housing in an open position and a fragrance disc disposed within a space;

FIG. 10 is a front elevational view of the decorative element of FIG. 9, shown in an assembled position; and,

FIG. 11 is a top plan view of the housing of the decorative element.

### DETAILED DESCRIPTION OF THE INVENTION

At the outset, it should be appreciated that like drawing numbers on different drawing views identify identical, or functionally similar, structural elements of the invention. It also should be appreciated that figure proportions and angles are not always to scale in order to clearly portray the attributes of the present invention.

While the present invention is described with respect to what is presently considered to be the preferred aspects, it is to be understood that the invention as claimed is not limited to the disclosed aspects. The present invention is intended to cover various modifications and equivalent arrangements included within the spirit and scope of the appended claims.

Furthermore, it is understood that this invention is not limited to the particular methodology, materials and modifications described and, as such, may, of course, vary. It is also understood that the terminology used herein is for the purpose of describing particular aspects only, and is not intended to limit the scope of the present invention, which is limited only by the appended claims.

Unless defined otherwise, all technical and scientific terms used herein have the same meaning as commonly understood to one of ordinary skill in the art to which this invention belongs. Although any methods, devices or materials similar or equivalent to those described herein can be used in the practice or testing of the invention, the preferred methods, devices, and materials are now described.

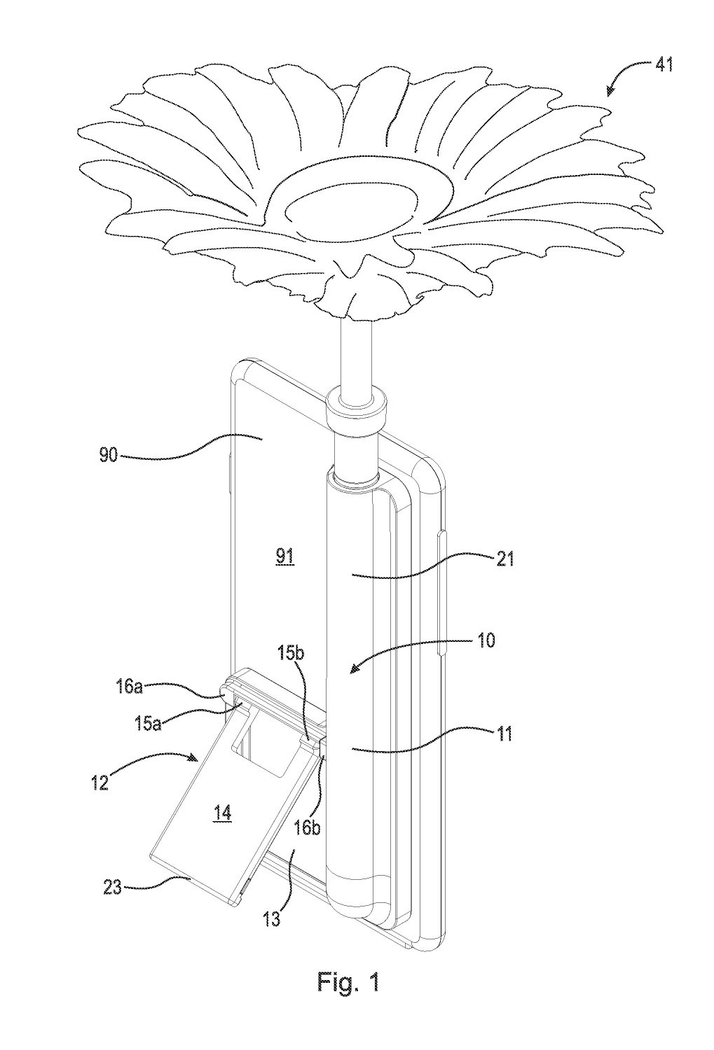

Adverting now to the figures, FIG. 1 is a front perspective view of a first embodiment of decorative accessory 10 of the present invention, shown removably secured to mobile device case 90.

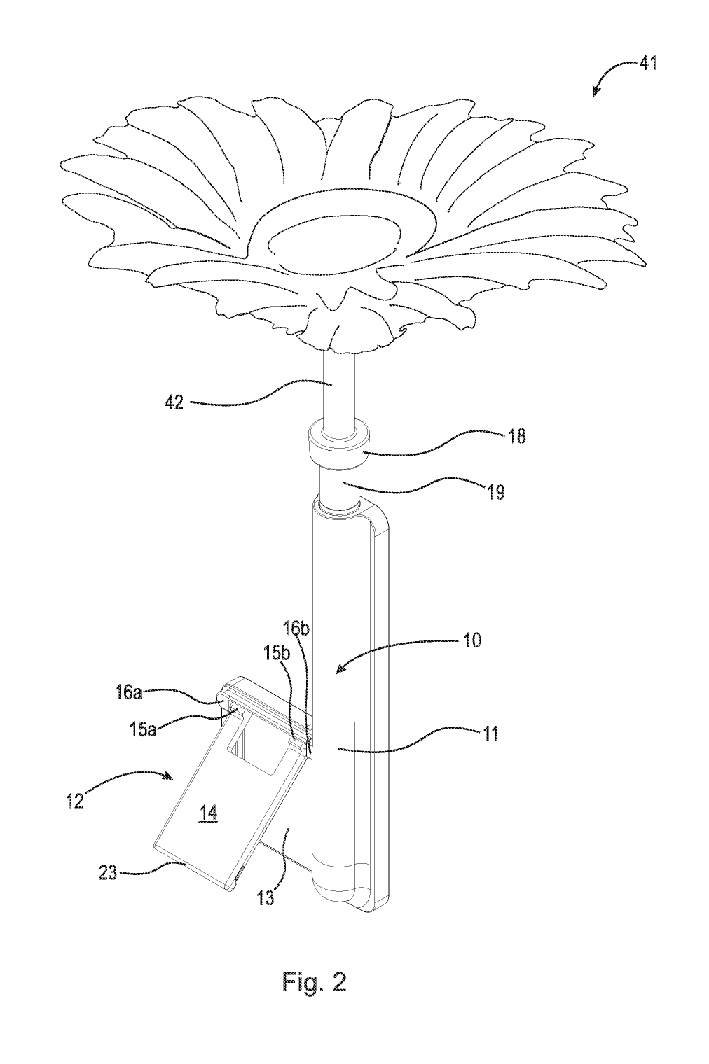

FIG. 2 is a front perspective view of the accessory of FIG. 1, shown with the case removed.

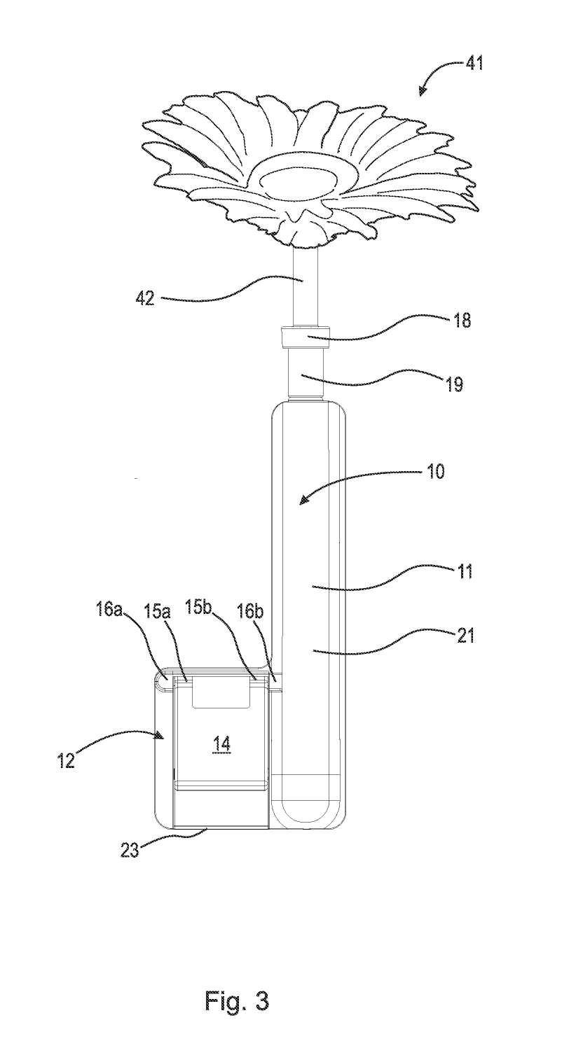

FIG. 3 is a front elevational view of the accessory of FIG. 2.

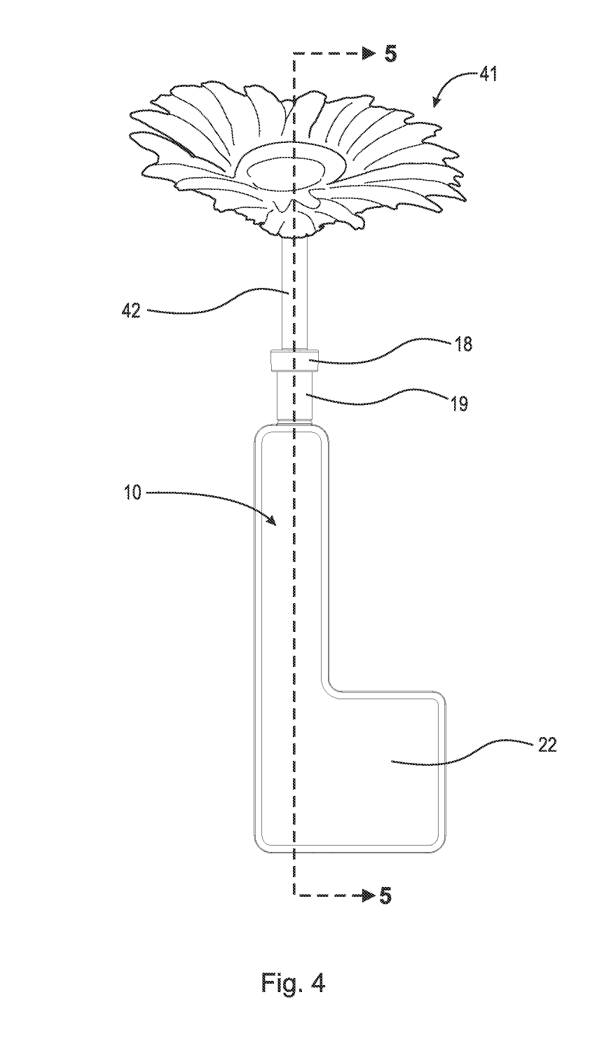

FIG. 4 is a rear elevational view of the accessory of FIG. 2.

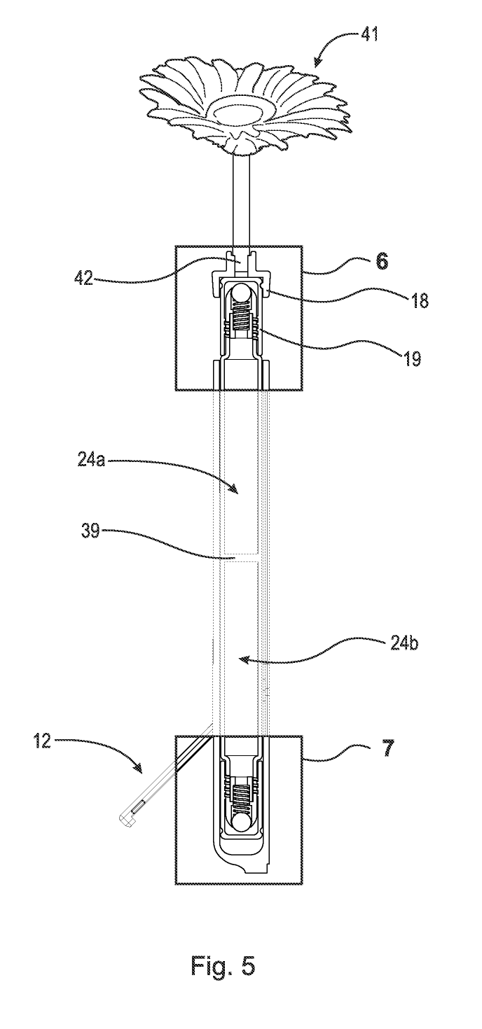

FIG. 5 is a cross-sectional view of the accessory, taken generally along line 5-5 of FIG. 4.

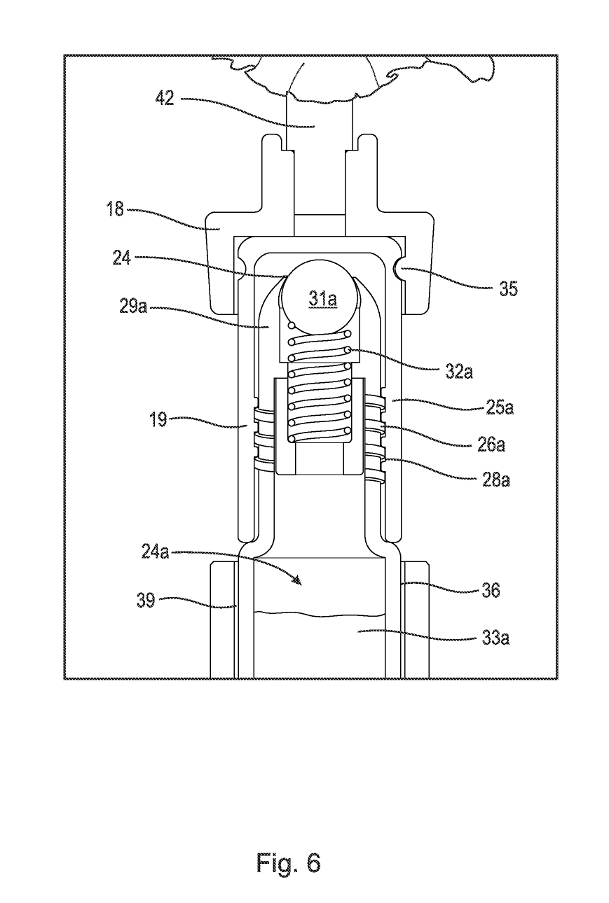

FIG. 6 is a detailed view of a first container of FIG. 5.

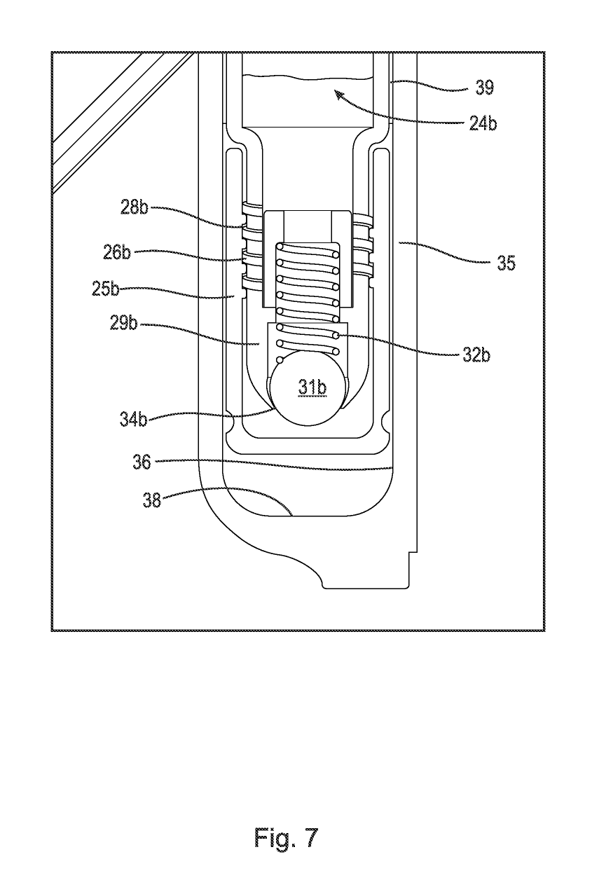

FIG. 7 is a detailed view of a second container of FIG. 5.

As shown in FIGS. 1 through 7, in the first embodiment, decorative accessory 10 includes substantially tubular-shaped portion 11, decorative element 41, and at least one cosmetic item 19. Tubular-shaped portion 11 has outer surface 21, inner surface 22, and cavity 39 bounded by inner surface 36. Decorative element 41 has extension member 42 extending downwardly therefrom, where extension member 42 is secured to tubular-shaped portion 11. At least one cosmetic item 19 is disposed within cavity 39. As shown in FIG. 1, mobile device case 90 includes surface 91 Inner surface 22 of tubular-shaped portion 11 is secured to surface 91 of mobile device case 90. Preferably, tubular-shaped portion 11 is secured via a removable adhesive; however, it should be appreciated that tubular-shaped portion 11 may be secured via any suitable securement means known in the art, such as adhesive, tape, glue, etc.

Preferably, decorative accessory includes two cosmetic items disposed opposite one another with cavity 39. Each cosmetic item includes container 24a, 24b, cap 25a, 25b, and fluid medium 33a, 33b disposed within container 24a, 24b, respectively. Preferably, the fluid medium is lip-gloss, lipstick, concealer, mascara, etc. However, it should be appreciated that the fluid medium may be any suitable medium known in art. Each cosmetic item further includes cap 25a, 25b, top portion 29a, 29b, ball 31a, 31b, spring 32a, 32b, and opening 34a, 34b, respectively. In particular, the first cosmetic item has first cap 25a, which includes first threads 26a, and first container 24a, which includes first grooves 28a. First cap 25a is threadably secured to first container 24a via first threads 26a and first grooves 28a. Similarly, the second cosmetic item has second cap 25b, which includes second threads 26b, and second container 24b, which includes second grooves 28b. Second cap 25b is threadably secured to first container 24b via first threads 26b and first grooves 28b. When the first cosmetic item is in use, first cap 25a is removed and first ball 31a is depressed into first opening 34a in order to allow first medium 33a to flow through first opening 34a. Similarly, when the second cosmetic item is in use, second cap 25b is removed and second ball 31b is depressed into second opening 34b in order to allow second medium 33b to flow through second opening 34b. When not in use, at least one cosmetic item 19 is disposed within cavity 19. In particular, second container 24b is inserted into cavity 39 first and rests upon inner bottom surface 38 of tubular-shaped portion 11. First container 24a is inserted second and rests upon second container 24b. Attachment means 18 includes notch 35, which is secured to first cap 25a.

In one embodiment, decorative accessory 10 further includes stand 12. Stand 12 has mount 13, flange 14, and at least one hinge 16a, 16b. Stand 12 extends laterally from tubular-shaped portion 11. Mount 13 is secured to a mobile device via mobile device case 90 and flange 14 is secured to and pivotable outwardly about at least one hinge 16a, 16b. Flange 14 includes first attachment means 15a, second attachment means 15b, and edge 23. First attachment means 15a is further secured to and pivotable about first hinge 16a. Similarly, second attachment means 15b is further secured to and pivotable about second hinge 16b. Flange 14 pivots outwardly into an open position, such that edge 23 rests upon a surface to support the mobile device in a substantially upright position and pivots inwardly into a closed position when not in use.

Decorative accessory 10 further includes attachment means 18 to secure extension member 42 of decorative element 41 to tubular-shaped portion 11. Additionally, decorative element 41 is substantially flower-shaped. Preferably, the decorative accessory is made of plastic and substantially tubular-shaped. However, it should be appreciated that any of the elements can be made of any suitable material known in the art and can be of any suitable shape known in the art.

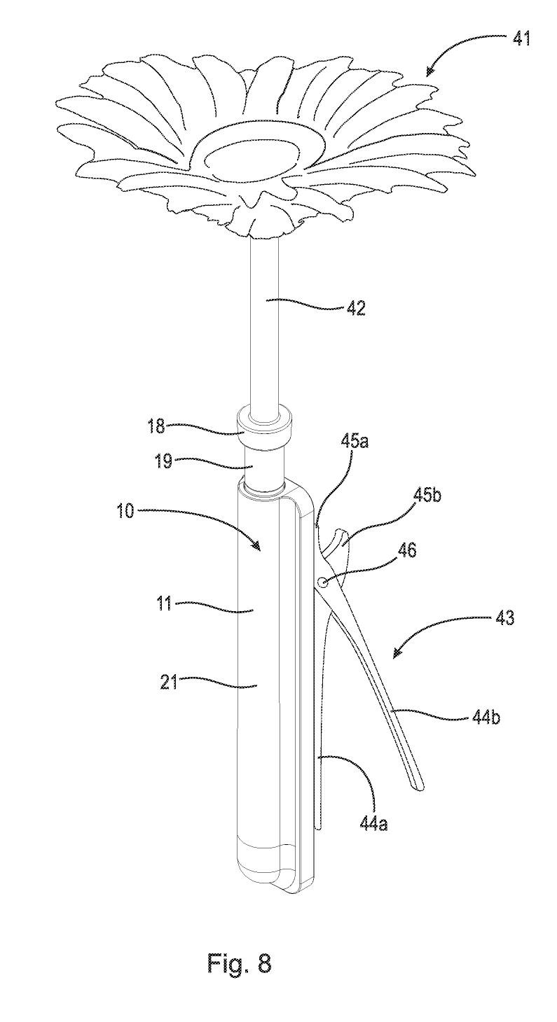

FIG. 8 is a second embodiment of decorative accessory 12, shown having clip 43 attached and with mobile device case 90 removed. In the second embodiment, decorative accessory 10 includes substantially tubular-shaped portion 11, decorative element 41, and at least one cosmetic item 19. Tubular-shaped portion 11 has outer surface 21, inner surface 22, and cavity 39 bounded by inner surface 36. Decorative element 41 has extension member 42 extending downwardly therefrom, where extension member 42 is secured to tubular-shaped portion 11. At least one cosmetic item 19 is disposed within cavity 39. Clip has first extension 44a, second extension 44b, first handle 45a, second handle 45b, and hinge 46. When second handle 45b is depressed, second extension 44b pivots outwardly about hinge 46 into an open position. Clip 43 is securable to an accessory when second extension 44b is released and pivots inwardly about hinge 46 into a closed position. It should be appreciated that clip 43 may be secured to any suitable accessory known in the art, such as a purse, backpack, pocket, etc.

Similar to the first embodiment, decorative accessory includes two cosmetic items disposed opposite one another with cavity 39. Each cosmetic item includes container 24a, 24b, cap 25a, 25b, and fluid medium 33a, 33b disposed within container 24a, 24b, respectively. Preferably, the fluid medium is lip-gloss, lipstick, concealer, mascara, etc. However, it should be appreciated that the fluid medium may be any suitable medium known in art. Decorative accessory 10 further includes attachment means 18 to secure extension member 42 of decorative element 41 to tubular-shaped portion 11. Additionally, decorative element 41 is substantially flower-shaped. Preferably, the decorative accessory is made of plastic and substantially tubular-shaped. However, it should be appreciated that any of the elements can be made of any suitable material known in the art and can be of any suitable shape known in the art.

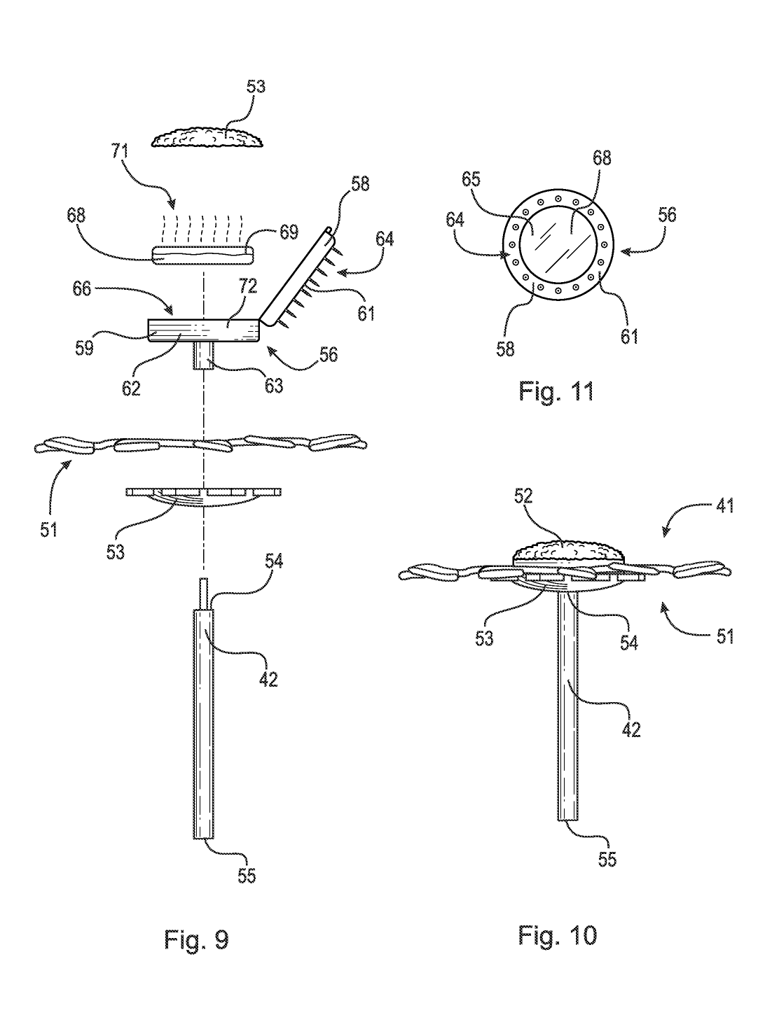

FIG. 9 is an exploded view of another embodiment of decorative element 13, in particular flower 41, shown having housing 56 in an open position and fragrance disc 68 disposed within cavity 66. Similarly, FIG. 10 is a front elevational view of flower 41, shown in an assembled and closed position. Moreover, FIG. 11 is a detailed top plan view of housing 56.

As shown in FIGS. 9 through 11, flower 41 broadly includes housing 56, fragrance disc 68, and means 63 for securing the decorative element to a mobile device or a case for a mobile device. Housing 56 has top 58, base 59, and attachment means 57. Attachment means 57 secures top 58 to base 59 and top 58 is pivotable about base 59. Base 59 has outer surface 72, inner surface (not shown), side 62 and cavity 66 bounded by side 62. Top 58 includes rim 61 and at least one aperture 65 bounded by rim 61. Fragrance disc 68 has an outer surface and an inner surface and is disposed within cavity 66 bounded by side 62 and fragrance disc 68 rests upon the inner surface of base 59. Fragrance disc 68 is filled with scented medium 69, which releases scent 71. Preferably, attachment means 57 is a hinge and means 63 for securing flower 41 to a mobile device extending downwardly from housing 56. Stem 42 of flower 41 has first end 54 and second end 55. First end 54 is secured to means 63 and second end 55 is secured to a mobile device, such as body 11 of cover 10.

Preferably, housing 56 is substantially circular in shape, in particular, top 58 is convex, and housing 56 is made of plastic. Preferably, decorative element 13 is substantially flower-shaped and can be of any flower variety. However, it should be appreciated that any of the elements can be made of any suitable material known in the art and can be of any suitable shape known in the art.

Top 58 further includes at least one barb 64 extending upwardly therefrom, and flower 41 further includes flower ovary 52 and plurality of petals 51. Flower ovary 52 is secured to at least one barb 64 and plurality of petals 51 are disposed circumferentially about base 59. Preferably, scented medium 69 is a perfume or an essential oil. However, is should be appreciated that the scented medium can be made of any suitable medium known in the art. Essential oils, such as lavender, have been thought to promote health benefits, such as soothing nervous tension, headaches, anxiety and depression, inducing sleep, relieving pain from soreness in the joints, disinfecting the scalp and skin, enhancing blood circulation, treating respiratory problems, and increasing the mobility of the intestine, as well as a variety of other health benefits.

In the various embodiments, the decorative element is substantially flower-shaped, spherically shaped, and in particular, a sports ball, or in the shape of an alligator. However, it should be appreciated that the decorative element can be any suitable shape known in the art. Furthermore, preferably, the body is made of plastic and substantially tubular-shaped. However, it should be appreciated that the body can be made of any suitable material known in the art and can be of any suitable shape known in the art. It should also be appreciated that, any of the fastening means of the cover can be any suitable fastening means known in the art, such as but not limited to, latches, snaps, hooks, etc.

Thus, it is seen that the objects of the present invention are efficiently obtained, although modifications and changes to the invention should be readily apparent to those having ordinary skill in the art, which modifications are intended to be within the spirit and scope of the invention as claimed. It also is understood that the foregoing description is illustrative of the present invention and should not be considered as limiting. Therefore, other embodiments of the present invention are possible without departing from the spirit and scope of the present invention.

What is claimed is:

1. A decorative accessory, comprising:
a substantially tubular-shaped portion, the tubular-shaped portion having an outer surface, an inner surface, and a cavity bounded by the inner surface;
a decorative element, the decorative element having an extension member extending downwardly therefrom, wherein the extension member is secured to the tubular-shaped portion; and,
at least one cosmetic item, wherein the at least one cosmetic item is disposed within the cavity.

2. The decorative accessory of claim 1, further comprising a stand, the stand having a mount, a flange, and at least one hinge, wherein the stand extends laterally from the tubular-shaped portion, the mount is secured to a device, and the flange is secured to and pivotable outwardly about the at least one hinge.

3. The decorative accessory of claim 1, further comprising an attachment means securing the extension member of the decorative element to the tubular-shaped portion.

4. The decorative accessory of claim 1, wherein the at least one cosmetic item comprises a container, a cap, and a fluid medium disposed within the container.

5. The decorative accessory of claim 4, further comprising two cosmetic items.

6. The decorative accessory of claim 1, wherein the decorative element is substantially flower-shaped.

7. A decorative accessory, comprising:
a substantially tubular-shaped portion, the tubular-shaped portion having an outer surface, an inner surface, and a cavity bounded by the inner surface;
a decorative element, the decorative element having an extension member extending downwardly therefrom, wherein the extension member is secured to the tubular-shaped portion;
at least one cosmetic item, wherein the at least one cosmetic item is disposed within the cavity; and,
a clip, the clip having a first extension, a second extension, a first handle, a second handle, and a hinge, wherein when the second handle is depressed, the second extension pivots outwardly into an open position.

8. The decorative accessory of claim 1, wherein the clip is securable about a device when the second extension is released and pivots inwardly into a closed position.

9. The decorative accessory of claim 1, further comprising an attachment means securing the extension member of the decorative element to the tubular-shaped portion.

10. The decorative accessory of claim 7, wherein the at least one cosmetic item comprises a container, a cap, and a fluid medium disposed within the container.

11. The decorative accessory of claim 10, further comprising two cosmetic items.

12. The decorative accessory of claim 7, wherein the decorative element is substantially flower-shaped.

13. A decorative element, comprising:
a housing, the housing having a top, a base, and an attachment means that secures the top to the base and the top is pivotable about the attachment means, the base having an outer surface, an inner surface, a side and a cavity bounded by the side, wherein the top includes a rim and at least one aperture bounded by the rim;
a fragrance disc, the fragrance disc having an outer surface and an inner surface, wherein the fragrance disc is disposed within the cavity bounded by the side and rests upon the inner surface of the base and the fragrance disc is filled with a scented medium; and,
a means for securing the decorative element to a mobile device.

14. The decorative element of claim 13, wherein the attachment means is a hinge.

15. The decorative element of claim 13, wherein the housing is substantially circular in shape.

16. The decorative element of claim 13, wherein the decorative element is substantially flower-shaped.

17. The decorative element of claim 16, wherein the means for securing the decorative element to a mobile device is an extension member extending downwardly therefrom.

18. The decorative element of claim 17, wherein the top further comprises at least one barb extending upwardly therefrom, and the decorative element further comprises a flower ovary and a plurality of petals, wherein the flower ovary is secured to the at least one barb and the plurality of petals are disposed circumferentially about the base.

19. The decorative element of claim 13, wherein the scented medium is an essential oil.

20. The decorative element of claim 13, wherein the top is substantially convex.
